(19) United States
(12) Patent Application Publication
(10) Pub. No.: US 2024/0410753 A1
(43) Pub. Date: Dec. 12, 2024

(54) SCREENING SYSTEM TO IDENTIFY PATHOGENS OR GENETIC DIFFERENCES

(71) Applicant: Avicena Systems Limited, West Perth (AU)

(72) Inventors: Anthony Stevens, West Perth (AU); Paul Watt, Mount Claremont (AU); Paul Ostergaard, Cottesloe (AU); Tatjana Heinrich, West Perth (AU); Robert Dewhurst, West Perth (AU)

(21) Appl. No.: 18/686,358

(22) PCT Filed: Aug. 25, 2022
(86) PCT No.: PCT/AU2022/051036

§ 371 Date: Feb. 23, 2024

(30) Foreign Application Priority Data
Aug. 25, 2021 (AU) 2021221694

(51) Int. Cl. G01J 3/10; G01J 3/44; G01J 3/50; G01N 21/25; G01N 21/64; G01N 35/00

(52) U.S. Cl. CPC G01J 3/10; G01J 3/4406; G01J 3/501; G01N 21/253; G01N 21/6452; G01N 35/0099; G01J 2003/104; G01J 2003/106; G01N 2021/6484; G01N 2035/00366; G01N 2201/0626

(57) ABSTRACT

A system for screening of pathogens or gene differences includes first and second screening modes and a source of electromagnetic radiation for illuminating a plurality of samples. The source of electromagnetic radiation has a selectable illumination property. The system also may include a detector for detecting electromagnetic radiation transmitted through or emitted by the plurality of samples with the detector having a selectable detection property. The system may be arranged for concurrent operation in the first and the second mode. The first screening mode may be a fluorometric screening mode and the second optional screening mode may be a colorimetric screening mode.

### CROSS REFERENCES

The application is a U.S. National Stage Application of International Application No. PCT/AU2022/051036, filed Aug. 25, 2022, which claims the benefit of priority based on Australian Patent Application No. 2021221694, filed on Aug. 25, 2021, the disclosures of each of which are hereby expressly incorporated by reference herein in their entireties.

### TECHNICAL FIELD

The present disclosure relates to a screening system to identify pathogens or genetic differences and relates particularly, though not exclusively, to a system for the detection of genetic differences, either in the DNA or RNA of genes, or in gene expression profiles.

### BACKGROUND

Especially the COVID-19 pandemic, but also other pandemics or epidemics require screening of large numbers of samples taken from symptomatic individuals who are expected to carry a virus or for routine surveillance screening of asymptomatic individuals in order to identify carriers of the virus. Different manual screening procedures are known, but in order to enable surveillance testing of larger numbers of samples, screening systems that enable higher throughput of samples are becoming more and more important.

Multiple sensitive and molecular diagnostic techniques exist for the detection of pathogens such as SARS-coV2 using a range of nucleic acid amplification and detection system, including the polymerase chain reaction (PCR), Isothermal Amplification methods and CRISPR based methods—for review reference is being made to [Habli, Z., Saleh, S., Zaraket, H. & Khraiche, M. L. COVID-19 in-vitro Diagnostics: State-of-the-Art and Challenges for Rapid, Scalable, and High-Accuracy Screening. Frontiers in Bioengineering and Biotechnology 8, (2021)].

A range of newer molecular tests are emerging which include, but are not limited to, technologies disclosed in the following publications:

Loop Mediated Isothermal Amplification (LAMP)—comprehensively reviewed here: [Moehling, T. J., Choi, G., Dugan, L. C., Salit, M. & Meagher, R. J. LAMP Diagnostics at the Point-of-Care: Emerging Trends and Perspectives for the Developer Community. Expert Rev Mol Diagn 21, 1-19 (2021)].

MD-LAMP [Becherer, L. et al. Simplified Real-Time Multiplex Detection of Loop-Mediated Isothermal Amplification Using Novel Mediator Displacement Probes with Universal Reporters. Anal Chem 90, 4741-4748 (2018)].

DETECTR [Broughton, J. P. et al. CRISPR—Cas12-based detection of SARS-COV-2. Nat Biotechnol 38, 870-874 (2020)].

miSHERLOCK [Puig, H. de et al. Minimally instrumented SHERLOCK (miSHERLOCK) for CRISPR-based point-of-care diagnosis of SARS-COV-2 and emerging variants. Sci Adv 7, eabh2944 (2021)]

SPOT. [Xun, G., Lane, S. T., Petrov, V. A., Pepa, B. E. & Zhao, H. A rapid, accurate, scalable, and portable testing system for COVID-19 diagnosis. Nat Commun 12, 2905 (2021)].

RTF-EXPAR [Carter, J. G. et al. Ultrarapid detection of SARS-COV-2 RNA using a reverse transcription-free exponential amplification reaction, RTF-EXPAR. Proc National Acad Sci 118, (2021)].

NACT [Moitra, P., Alafeef, M., Dighe, K., Frieman, M. B. & Pan, D. Selective Naked-Eye Detection of SARS-COV-2 Mediated by N Gene Targeted Antisense Oligonucleotide Capped Plasmonic Nanoparticles. Acs Nano 14, 7617-7627 (2020); Alafeef, M., Moitra, P., Dighe, K. & Pan, D. RNA-extraction-free nano-amplified colorimetric test for point-of-care clinical diagnosis of COVID-19. Nat Protoc 16, 3141-3162 (2021)].

Those skilled in the art will be aware that the reaction products of these molecular diagnostics assays can be detected through changes in colour (detected by differences in absorbance reflectance or transmission of illuminated light), luminescence phosphorescence or fluorescence.

One promising technique used for screening molecular signatures in samples is the so-called “Loop Mediated Isothermal Amplification (“LAMP”) technique. The screening process involves collecting biological samples (such as, but not limited to saliva, sputum, anterior nasal, mid-turbinate, or nasopharyngeal swabs and throat swabs), and placing the samples into test tubes, together with chemicals used for the LAMP process. The samples are then incubated and colorimetric or fluorometric detection techniques may be used to determine an outcome of the screening process. LAMP has the advantage that the incubation and the detection process can take as little as 20 to 30 minutes. Screening systems may be used for parallel processing and screening of samples thereby increasing the throughput compared with manual LAMP procedures.

However, to date the molecular diagnostics methods disclosed above, are currently implemented in low-throughput point-of-care formats, or in medium formats, without a feasible and economic means to operate at an ultra high-throughput scale. This means the standard approaches to molecular diagnostics disclosed above are not applicable to ultra high-throughput screening methods, particularly those methods supporting continuous operation at several thousand tests per hour. For example, even costly, high throughput molecular diagnostics instruments such as the Roche Cobas 6800, the Abbott Alinity, the Quiagen QIAstat-Dx, NeuMoDx or the Hologic Panther instruments, some of which support more continuous flow loading modes are not configured in a manner which allows economical scaling to continuous ultra-high throughput operation due to inherent design constraints.

Point of care solutions linked to small molecular assay devices and/or to smart phones also have their own limitations in ID verifiability, integration and affordable costs for implementation at the population scale or in biosecurity surveillance applications.

Accordingly, the ability to rapidly screen very large number of samples associated with a pandemic or to screen economically for genetic changes at the population level in minimum timeframes, requires not only parallel processing of the samples at ultra-high throughput, but also requires further technical solutions for increasing throughput and versatility, allowing flexible adaptation for fluctuations in testing volumes such as, but not limited to scalable random access, continuous flow loading. There is a need for technological advancement.

### SUMMARY

An embodiment relates to technology that enables configuration for screening in a variety of distinct modes (eg. fluorescence and colorimetric modes), using inexpensive components which are less subject to supply chain constraints in a pandemic.

The inventors have found that a key limitation of applying standard molecular diagnostic approaches to ultra-high throughput screening is a requirement for rapid changing of a combination of excitation and emission filters in a continuously scanning fluorescent detection system, while avoiding complex and costly synchronisation approaches. Therefore, existing approaches and configurations used for high-throughput screening are not applicable to ultra high-throughput screening. For example, the need to change filters to detect emissions from diagnostic fluorophore probes, and synchronise such filters with excitation sources increases complexity and costs, limits throughput rates to a few thousand samples per hours (e.g., <1000 samples/hour). The term “ultra high-throughput” as used herein means a system capable of screening with a continuous operation of least 2000 samples per hour.

The present disclosure provides in a first aspect a screening system to identify pathogens or genetic differences, wherein the system can be configured to support first and/or second screening modes and comprising:
a source of electromagnetic radiation for illuminating a plurality of samples, the source of electromagnetic radiation having a selectable illumination property; and
a detector for detecting electromagnetic radiation transmitted through or emitted by the plurality of samples, the detector having a selectable detection property; and
an incubator for incubating the samples;
wherein the system is arranged for operation in the first and second screening mode during incubating of the samples in the incubator.

The present disclosure provides in a first aspect a screening system to identify pathogens or genetic differences, wherein the system can be configured to support first and/or second screening modes and comprising:
a source of electromagnetic radiation for illuminating a plurality of samples, the source of electromagnetic radiation having a selectable illumination property; and
a detector for detecting electromagnetic radiation transmitted through or emitted by the plurality of samples, the detector having a selectable detection property; and
an incubator for incubating the samples;
wherein the system is arranged for operation in the first and second screening mode during incubating of the samples in the incubator.

The system may be arranged for concurrent or quasi-concurrent operation in the first and in the second mode.

The present disclosure provides in a second aspect a screening system to identify pathogens or genetic differences, wherein the system can be configured for first and second screening modes and comprises:
a source of electromagnetic radiation for illuminating a plurality of samples, the source of electromagnetic radiation having a selectable illumination property; and
a detector for detecting electromagnetic radiation transmitted through or emitted by the plurality of samples, the detector having a selectable detection property;
wherein the system is arranged for concurrent operation in the first and the second mode.

The system may comprise an arrangement for processing the samples, which may be an incubator.

The system typically is arranged for operation in the first and/or second screening mode during incubating of the samples in the incubator.

The following embodiments introduces examples of optional features of the system in accordance with the first or second aspect of the present disclosure.

In one embodiment, the system is an ultra high-throughput system. In an embodiment, the system is configured to process at least 2,000 samples per hour. For example, the system may be configured to process in an hour at least 2,500 samples, 3,000 samples, 3,500 samples, 4,000 samples, 4,500 samples, 5,000 samples, 5,500 samples, 6,000 samples, 6,500 samples, 7,000 samples, 7,500 samples, 8,000 samples, 8,500 samples, 9,000 samples, 9,500 samples, or 10,000 samples. In an embodiment, the system is configured to process from about 4,000 samples to about 10,000 samples per hour.

In one embodiment the first screening mode is a fluorometric screening mode and the second screening mode is a colorimetric screening mode. Alternatively, the first or second mode may be a luminescence or phosphorescence screening mode. In one embodiment the first screening mode is a first fluorometric screening mode and the second screening mode is a second fluorometric screening mode. The system may include a third or higher screening mode. For example, the system may comprise first, second and third screening modes. Regardless of the number of screening modes, the modes are arranged for concurrent or quasi-concurrent operation.

In an embodiment, the source of electromagnetic radiation and/or the detector has an associated fixed optical filter or filters. In an embodiment, components of an optical system associated with the source of electromagnetic radiation and the detector is configured to remain static or fixed in use of the system. For example, in an embodiment, optical filters used in the optical system remain fixed during use of the system. Put another way, in an embodiment, there is no requirement for adjustment or changing of optical filters during use of the system. This can help to reduce or eliminate the need to synchronise the source of electromagnetic radiation and the detector, which can help to reduce complexity and cost of the system and help to increase throughput rates. In comparison, high-throughput systems (i.e., those that process <1000 samples/hour) typically require switching and synchronisation of optical filters, which adds capital costs, lowers feasible throughput rates, adds complexity, and increases running costs.

The system may be arranged such that screening conditions can be changed in an automated manner or in accordance with a predetermined screening protocol which may be modulated by a controller. Change of the screening conditions may be effected by selecting at least one of: the illumination property of the source of electromagnetic radiation and the detection property of the detector.

Further, the system may be arranged such that individual samples or individual groups of samples are concurrently or quasi-concurrently screened using different conditions. For example, a first individual sample or a first individual group of samples may be screened using the first screening mode such as the fluorometric screening mode while concurrently or quasi-concurrently a second individual sample or second individual group of samples may be screened using the second screening mode such as the colorimetric screening mode.

In one embodiment the arrangement for processing samples allows processing and/or screening of groups of samples using different conditions (such as one or more of: heat treatment, illumination conditions, detection conditions). More specifically, the arrangement for processing samples may allow illuminating individual samples or groups of samples using different conditions such as conditions required for the first (e.g., fluorometric) screening mode or the second (e.g., colorimetric) screening mode.

The arrangement for processing the samples may be suitable for holding and processing a large number of samples, such as a few hundred or thousand samples. The arrangement for processing the samples may comprise individual sample holders and may comprise groups or arrays of individual sample holders, such as groups, combinations or arrays of 1-12, 12-24, 24-28, 48-96 or more of individual sample holders. The arrangement for processing the samples may comprise any suitable number of the groups of sample holders, such as 1-4, 4-8, 8-12, 12-16, 16-20, 20-24 or more.

The system may further include a sample vessel, which may include one or more cavities for receiving samples. Examples of sample vessels include capillaries or tubes (which may be held in racks of a transparent material) and microplates with cavities, such as 96 cavities, for receiving the samples and which may contain chemicals required for screening and/or processing of the samples. The cavities of the sample vessel may be sealed.

In one specific embodiment the cavities of the sample vessel include an amount of oil or low melting temperature wax, comprised of paraffin (eg. mineral oil or paraffin wax) or alternatively comprised of silicone wax. The inventors have observed that the presence of the oil in the cavities has advantages for screening and processing of the samples. The presence of the oil (such as an oil layer over each sample) may increase the quality of results from colorimetric and fluorometric RT-LAMP reactions, may provide a seal for the samples blocking unwanted aeration of the reaction mixes thereby avoiding that reaction mixes spontaneously acidify during storage as well as avoiding evaporation of the reaction mixes during incubation and may reduce likelihood of false positives when screening samples in accordance with embodiments of the present disclosure. The melting temperature of any wax layer can be adjusted to ensure that this layer of wax liquifies for operation in the instrument.

Further, the arrangement for processing the samples may comprise heaters and one or more controller enabling individual control of heating of individual samples or individual groups of samples.

Those skilled in the art will be aware that the heaters may comprise isothermal heating units operating at constant temperature (suitable for chemistries such as RT-LAMP) and may alternatively or also comprise thermal cycler units (suitable for chemistries such as PCR).

Further, those skilled in the art will also appreciate that independent control of the heaters will enable temperature changes or transfer of sample vessels such as microplates from one temperature zone of the instrument for one part of the reaction (eg. RT-LAMP reaction) to another zone for another activity (such as for an additional incubation at a distinct temperature or for measuring melting/reannealing kinetics). This feature is ideal for CRISPR based technologies which incorporate two distinct incubation temperatures.

The system may further comprise a robotic system for loading and unloading of samples. The system for screening of pathogens is typically arranged in order to identify if and when the screening and/or processing is completed for individual samples or groups of samples, such as samples in individual microplates. The robotic system then removes the individual samples or groups of samples (or sample vessels containing samples, such as microplates with samples contained within wells therein), which may be at random positions within the arrangement for processing samples and may be surrounded by, or adjacent to, samples (or sample vessels with samples such as microplates with samples) for which the screening and/or processing is not yet completed, whereby vacant positions in the arrangement for processing samples are generated. The robotic system is then arranged to obtain fresh samples or groups of fresh samples (or microplates with fresh samples), for example from a sample waiting station, and to fill the vacant positions in the arrangement for processing samples with the fresh samples. In this manner the system for screening pathogens or genetic changes in accordance with an embodiment of the present disclosure is suitable for continuous throughput of samples, which facilitates very high throughput operation not possible with a batch processing technique. This continuous throughput design also offers more economical operation than previous attempts at high-throughput operation which are only economical at high loading volumes. By contrast the screening system described here can be equally loaded with a single unit of samples (such as a 96 or 384 well microplate) as with a plurality of such samples units at any interval greater than the minimum loading cycle time of the instrument. The minimal loading cycle time may be approximately 2 minutes. The minimal loading cycle time may be approximately 1 minute. The minimal loading cycle time may be less than one minute.

The flexibility of the system disclosed here allows for completely independent reaction chemistries to be run in parallel, for example an RT-PCR reaction to be run in one part of the instrument incubation zone, while an RT-LAMP reaction is run in another part of the instrument incubation zone.

In one example the illumination property is a light intensity and/or a wavelength or wavelengths range of the electromagnetic radiation. The source of electromagnetic radiation may comprise a number of component sources for emitting largely monochrome electromagnetic radiation, and may include one or more of the following: light emitting diodes (LED); tuneable laser(s); optical filter(s) and/or mirror(s); and dichroic filter(s). If the source of electromagnetic radiation is arranged to emit light at different wavelengths, the components may be selected and/or adjusted to select a wavelength or wavelength range of electromagnetic radiation emitted by the source of electromagnetic radiation.

In one embodiment the source of electromagnetic radiation comprises a light source for the fluorometric mode (such as LEDs or lasers) and a light source for the colorimetric mode. In one embodiment, the source of electromagnetic radiation comprises a first light source for a first fluorometric mode and a second light source for a second fluorometric mode. The source of electromagnetic radiation may comprise a broadband light source which may have suitable filters and which may be suitable for illumination in the colorimetric mode. The source of electromagnetic radiation may be arranged for illumination of the samples from a position over (above) or below the samples or from a horizontal direction.

In one example the source of electromagnetic radiation comprises individual light elements, such as LEDs and individual LEDs or groups of LEDs with filters may be positioned at respective sample holders for direct illumination of the samples. Alternatively, or in addition the source of illumination may comprise a diffuser to which individual light elements, such LEDs with filters are coupled and which are arranged to generate diffuse light for illuminating samples for screening of the samples in the first and/or second screening mode.

Alternatively or additionally, the system may also comprise optical fibres between the source of electromagnetic radiation and individual sample holders or groups of the sample holders. The optical fibres may be guided through portions of the arrangement, for example through a collimator element for processing the samples to the individual sample holders or to groups of the sample holders. In one embodiment the source of light may be one or more a switchable variable laser light sources, liked via a collimator element to a bundle of optical fibres linked to the sample holders or groups of sample holders.

In one example the detection property is a wavelength or wavelengths range of the electromagnetic radiation detectable by the detector, which may be selectable by selecting a filter.

The detector may be arranged for detecting electromagnetic radiation at different wavelengths (or wavelengths ranges) providing wavelength specific information signals (such as a colour camera showing a colour). The detector may for example comprise a colour camera, a monochrome detector such as a monochrome camera, or scanning arrays of photodiodes or photomultipliers. The detector may include a multi-pass filter or a bandpass filter.

The detector may comprise a single detection component or multiple detection components each providing signals as a function of detected light intensity. The detector may also be one of a plurality of detectors. In one embodiment at least two detectors are arranged to generate signals largely independent of a wavelength of electromagnetic radiation within a given wavelengths range (“monochrome detector”), such as a monochrome camera. In this example each detector may comprise one or more selectable filters which may optionally remain fixed in operation, such as filters allowing the transmission of electromagnetic radiation at a selected wavelengths range while at least partially blocking transmission of electromagnetic radiation at other wavelengths ranges whereby it is possible to detect electromagnetic radiation at different wavelength or wavelengths ranges (as properties of the used filter are known). It is consequently possible to use the monochrome detectors for detecting electromagnetic radiation associated with the fluorometric mode or the colorimetric mode. For example, suitable long-pass or bandpass or multi-pass filters may be used for this purpose. In this example a first detector may be arranged to operate in a fluorometric mode and a second detector may be arranged to operate simultaneously or in rapid succession in a colorimetric mode. In another example, if the system operates with a first, second and third fluorometric screening mode, a first colour or monochrome camera can be used to detector emission from the first fluorometric screening mode, a second colour or monochrome camera can be used to detector emission from the second fluorometric screening mode, and a third colour or monochrome camera can be used to detector emission from the third fluorometric screening mode. The first, second and third cameras can be grouped together to form a “stack” or may operate independent of one another.

In one specific embodiment the system comprises optical fibres between the detector and each individual sample holder or group of the sample holders for receiving samples. The optical fibres may be positioned to receive radiation from the samples (such as excited fluorescent radiation for fluorometric screening or transmitted or reflected radiation for colorimetric screening) and direct the received radiation to a suitable detection element (such as a computer-controlled camera). In one variation of this embodiment the source of electromagnetic radiation is also optically coupled to individual samples via optical fibres and both the detector and the source of electromagnetic radiation may be coupled to the same optical fibre portions using a dichroic combiner/splitter.

The detector may be moveable to detect electromagnetic radiation at a location near an individual sample or group of individual samples. The movement of the detector may be controlled by a controller.

The present disclosure provides in a third aspect a screening system to identify pathogens or genetic differences, wherein system has first and second screening modes and comprises:
a source of electromagnetic radiation for illuminating a plurality of samples, the source of electromagnetic radiation having a selectable illumination property; and
a detector for detecting electromagnetic radiation transmitted through or emitted by the plurality of samples, the detector having a selectable detection property; and
an arrangement for processing the samples;
wherein the system is arranged for transferring between the first screening mode and the second screening mode by selecting at least one of the detection property of the detector and the illumination property of the source of electromagnetic radiation.

The system may enable operation in one of the first and second mode immediately after operation in the other one of the first and second mode and typically during incubation.

In one embodiment the first screening mode is a fluorometric screening mode and the second screening mode is a colorimetric screening mode. Alternatively, the first or second mode may be a luminescence or phosphorescence screening mode. This first and second screening modes may be a first fluorometric screening mode and a second fluorometric screening mode.

The arrangement for processing the samples typically is an arrangement for incubating the samples.

The system may be arranged such that screening conditions can be changed in an automated manner or in accordance with a predetermined screening protocol which may be controlled by a controller. Change of the screening conditions may be effected by selecting at least one of: the illumination property of the source of electromagnetic radiation and the detection property of the detector.

Further, the system may be arranged such that individual samples or individual groups of samples are screened using different conditions. For example, a first individual sample or a first individual group of samples may be screened using the first screening mode such as the fluorometric screening mode while a second sample or second individual group of samples is screened using the second screening mode such as the colorimetric screening mode.

In one embodiment the arrangement for processing samples allows processing and/or screening of groups of samples using different conditions (such as one or more of: heat treatment, illumination conditions, detection conditions). More specifically, the arrangement for processing samples may allow illuminating individual samples or groups of samples using different conditions such as conditions required for the fluorometric screening mode or the colorimetric screening mode such illumination may occur simultaneously or in rapid succession.

The arrangement for processing the samples may be suitable for holding and processing a large number of samples, such as a few hundred or thousands of samples. The arrangement for processing the samples may comprise individual sample holders and may comprise groups or arrays of the individual sample holders, such as a groups or arrays of 1-12, 12-24, 24-28, 48-96 or more individual sample holders. The arrangement for processing the samples may comprise any suitable number of the groups of individual sample holders, such as 1-4, 4-8, 8-12, 12-16, 16-20, 20-24 or more.

The system may further include a sample vessel, which may include one or more cavities for receiving samples. Examples of sample vessels capillaries or tubes (which may be held in racks of a transparent material) and microplates with cavities, such as 96 cavities, for receiving the samples and which may contain chemicals required for screening and/or processing of the samples. The cavities of the sample vessel may be sealed. In one specific embodiment the cavities of the sample vessel include an amount of oil, such as a mineral oil. The inventors have observed that the presence of the oil in the cavities has advantages for screening and processing of the samples. The presence of the oil (such as an oil layer over each reaction well in the reaction vessel) may increase the quality of results from colorimetric and fluorometric RT-LAMP reactions, may provide a seal for the samples blocking unwanted aerosol contamination or evaporation of the reaction mixes thereby avoiding that reaction mixes become more concentrated, and may reduce likelihood of false positives when screening samples in accordance with embodiments of the present disclosure.

Further, the arrangement for processing the sample reactions may comprise heaters and one or more controller enabling individual control of heating of individual reactions or individual groups of reactions.

Those skilled in the art will be aware that the heaters may comprise isothermal heating units operating at constant temperature (suitable for chemistries such as RT-LAMP) or thermal cycler units (suitable for chemistries such are PCR) capable of rapidly changing temperature, for example via Peltier-effect or magnetic induction temperature ramping methods.

Further, those skilled in the art will also appreciate that independent control of the heaters will enable temperature changes or transfer of sample vessels such as microplates from one temperature zone of the instrument for one part of the reaction (eg. RT-LAMP reaction) to another zone for another activity (such as measuring melting/reannealing kinetics or for DNA sequencing—in the case of LAMPseq protocols, for example.

The system may further comprise a robotic system for rapid loading and unloading of samples into the reaction instrument. The system for screening of pathogens is typically arranged to identify if and when the screening and/or processing has been completed for individual sample reactions or groups of reactions, such as samples in individual microplates. The robotic system then removes the individual reactions or groups of reactions (or sample vessels with samples such as microplates with samples), which may be at random positions within the arrangement for processing samples and may be surrounded by, or adjacent to, samples (or sample vessels with samples such as microplates with samples) for which the screening and/or processing is not yet completed whereby vacant positions in the arrangement for processing samples are generated. The robotic system is then arranged to obtain fresh samples and reaction vessels or groups of fresh samples (and/or microplates with fresh samples), for example from a sample waiting station, and to fill the vacant positions in the arrangement for processing samples with the fresh samples. In this manner the system for screening pathogens in accordance with an embodiment of the present disclosure is suitable for continuous throughput loading of samples, or semi-continuous loading at random intervals within the minimum loading cycle time, which facilitates very high throughput operation not possible with a batch processing technique. An important consequence of treating individual sample reactions independently is that they can be incubated scanned and analysed independently, despite being incubated and scanned together with a diverse array of other sample reactions, the results of which are deconvoluted afterwards with regards to their origin.

In one example of detection modalities, the detection property is a wavelength or wavelengths range of the electromagnetic radiation detectable by the detector. The detector may for example comprise a colour camera, a monochrome detector such as a monochrome camera, or scanning arrays of photodiodes or photomultipliers.

The detector may comprise a single detection component or multiple detection components each providing signals as a function of detected light intensity.

In one specific embodiment the detector is arranged to generate a signal largely independent of a wavelength of electromagnetic radiation with a given wavelength range (“monochrome detector”), such as a monochrome camera. In this example the detector may comprise one or more selectable filters, such as filters allowing the transmission of electromagnetic radiation at a selected wavelengths range while at least partially blocking transmission of electromagnetic radiation at other wavelengths ranges whereby it is possible to detect electromagnetic radiation at different wavelength and identify the colour (as properties of the used filter are known) using a monochrome detector. For example, suitable long-pass or bandpass filters or multi-pass filters may be used. Alternatively, or in addition the detector may be arranged for detecting electromagnetic radiation simultaneously at different wavelengths or wavelengths ranges, providing wavelength specific information (such as a via colour camara or photomultiplier array detecting a particular spectrum of colours).

In one embodiment the detector is a monochrome detector and comprises filters which allow the transmission of electromagnetic radiation for either the fluorometric or the colorimetric mode, but blocking other radiation at another wavelength range. For example, a first filter may allow transmission of fluorescence radiation at a specific wavelengths range and a second filter may allow transmission of electromagnetic radiation at a wavelengths range required for the colorimetric mode. By transferring between the first and second filter, the system may be transferred between the fluorometric mode and the colorimetric mode and the fluorometric and colorimetric measurements are possible in sequence. As the transfer between the first and second filters can take place within a short period of time, the system enables immediate transfer between the fluorometric mode and the colorimetric mode.

In a variation of the above-described embodiment the detector comprises a multi-pass filter which allow the transmission of electromagnetic radiation in first and second wavelengths ranges wherein the first wavelength range may be suitable for detection in one fluorometric mode and the second wavelength range may be suitable for detection in another fluorometric mode while blocking other radiation at another wavelength range. By transferring between illumination suitable for one fluorometric mode and illumination suitable for the other fluorometric mode, the system may be transferred between the distinct fluorometric modes and the different fluorometric measurements are possible in sequence. As the transfer between the illumination suitable for one (fluorometric) mode and illumination suitable for the other fluorometric mode can take place within a short period of time, the system enables immediate transfer between the distinct fluorometric modes. Those skilled in the art will be aware that such a multiplex detection capability can be used for distinguishing a number of distinct reaction products present in each in the same incubation vessel.

Further, the monochrome detector may be arranged for ratiometric intensity measurement. For example, the ratiometric intensity measurement may require illumination of samples at a first wavelengths range and at a second wavelengths range. By selecting the illumination at the first wavelengths range and subsequently illumination at the second wavelength range and detecting respective light intensities using the monochrome detector, ratiometric intensity measurement are possible using the monochrome detector.

In one example the illumination property is a light intensity and/or a wavelength or wavelengths range of the electromagnetic radiation. The source of electromagnetic radiation may comprise a number of component sources for emitting largely monochrome electromagnetic radiation, such as light emitting diodes (LED) which are arranged to emit light at different wavelengths and which may be selectable to select a wavelength or wavelength range of electromagnetic radiation emitted by the source of electromagnetic radiation.

In one specific embodiment the source of electromagnetic radiation comprises a narrow spectrum light source for the fluorometric mode (such as LEDs or lasers) and a light source for the colorimetric mode which may span a broader band of wavelengths such as white light. The source of electromagnetic radiation may comprise a broadband light source which may have suitable filters and or diffraction gratings and/or prisms and may be suitable for both fluorometric and/or for colorimetric screening. The source of electromagnetic radiation may be arranged for illumination of the samples from a position over or below the samples or from a horizontal direction.

In one example the source of electromagnetic radiation comprises individual light elements, such as LEDs, and individual LEDs or groups of LEDs with filters which may be positioned at respective sample holders for direct illumination of the samples. Alternatively, the source of illumination may comprise a diffuser to which individual light elements, such LEDs with one or more filters are coupled and which are arranged to generate diffuse light for illuminating at least groups of samples for screening of the samples in the first and/or second screening mode.

Alternatively or additionally, the screening system may also comprise optical fibres between the source of electromagnetic radiation and individual sample holders or groups of the sample holders. The optical fibres may be guided through portions of the arrangement for processing the samples to the individual sample holders or to groups of the sample holders.

In one specific embodiment the system comprises optical fibres between the detector and each individual sample holder for receiving a sample or group of the sample holders. The optical fibres may be positioned to receive radiation from the samples (such as excited fluorescent radiation for fluorometric screening or transmitted radiation for colorimetric screening) and direct the received radiation to a suitable detection element (such as a computer-controlled camera). In one variation of this embodiment the source of electromagnetic radiation is also coupled to individual samples via optical fibres and both the detector and the source of electromagnetic radiation may be optically coupled to the same optical fibre portion using a dichroic combiner/splitter in optional combination with a collimator element.

Further, in another embodiment a colour detector such as a colour camera may be arranged for ratiometric intensity measurement. For example, the ratiometric intensity measurement may require illumination of samples at a first wavelengths range and at a second wavelengths range. By selecting the illumination at the first wavelengths range and subsequently illumination at the second wavelength range and detecting respective light intensities using the colour detector, ratiometric intensity measurement are possible using a colour detector.

The source of electromagnetic and the detector of the first and/or second aspects may be used in the third aspect.

The detector may be moveable to detect electromagnetic radiation at a location near an individual sample or group of individual samples. The movement of the detector may be controlled by a controller.

Disclosed is a method of identify pathogens or genetic differences using a first and second screening mode, comprising:
incubating a plurality of samples, and
using the first and second screening mode during incubation by;
illuminating the plurality of samples with a source of electromagnetic radiation having a selectable illumination property;
detecting electromagnetic radiation transmitted through or emitted by the plurality of samples with a detector having a selectable detection property.

The first and second screening modes may be used concurrently or quasi-concurrently during incubation. Illuminating and detecting during incubation can help to increase a sample throughput rate. The source of electromagnetic radiation may include different sources of electromagnetic radiation. The source of electromagnetic radiation may be as described above in the first, second and/or third aspects. The detector having a selectable detection property may include different detectors. The detectors may be as described above in the first, second and/or third aspects. In an embodiment, optical filters associated with the source of electromagnetic radiation and/or the detector remain fixed. The method may otherwise be performed as described above for use of the system of the first, second and/or third aspects.

These and other aspects are merely illustrative of the innumerable aspects associated with the present disclosure and should not be deemed as limiting in any manner. These and other aspects, features, and advantages of the present disclosure will become apparent from the following detailed description when taken in conjunction with the referenced drawings.

### BRIEF DESCRIPTION OF THE DRAWINGS

Reference is now made more particularly to the drawings, which illustrate the best presently known mode of carrying out the present disclosure and wherein similar reference characters indicate the same parts throughout the views.

FIGS. 1 to 5 are schematic representation of systems screening systems to identify pathogens or genetic differences in accordance with embodiments of the present disclosure;

FIG. 6 is a component of an arrangement for holding and incubating samples in accordance with an embodiment of the present disclosure;

FIG. 7(a) is a source of electromagnetic radiation in accordance with an embodiment of the present disclosure;

FIG. 7(b) is a cross-sectional representation of an optical fibre bundle in accordance with an embodiment of the present disclosure; and

FIG. 8 is a graph of false positives as a function of incubation time for processing samples using a system in accordance with embodiment of the present disclosure.

### DETAILED DESCRIPTION OF EMBODIMENTS

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

In the following detailed description numerous specific details are set forth in order to provide a thorough understanding of the present disclosure. However, it will be understood by those skilled in the art that the present disclosure may be practiced without these specific details. For example, the present disclosure is not limited in scope to the particular type of industry application depicted in the figures. In other instances, well-known methods, procedures, and components have not been described in detail so as not to obscure the present disclosure.

The headings and sub-headings used herein are intended only for general organization of topics within the present disclosure and are not intended to limit the disclosure of the technology or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. All references cited in the “Detailed Description” section of this specification are hereby incorporated by reference in their entirety.

Embodiments of the present disclosure relate to a screening system to identify pathogens or genetic differences. The system is highly configurable and enables high-throughput colorimetric and/or fluorometric screening of the pathogens in a concurrent, quasi-concurrent or sequential manner. The screening can be conducted in accordance with testing parameters as required by desired test protocols and the pathogens being detected in an automated manner.

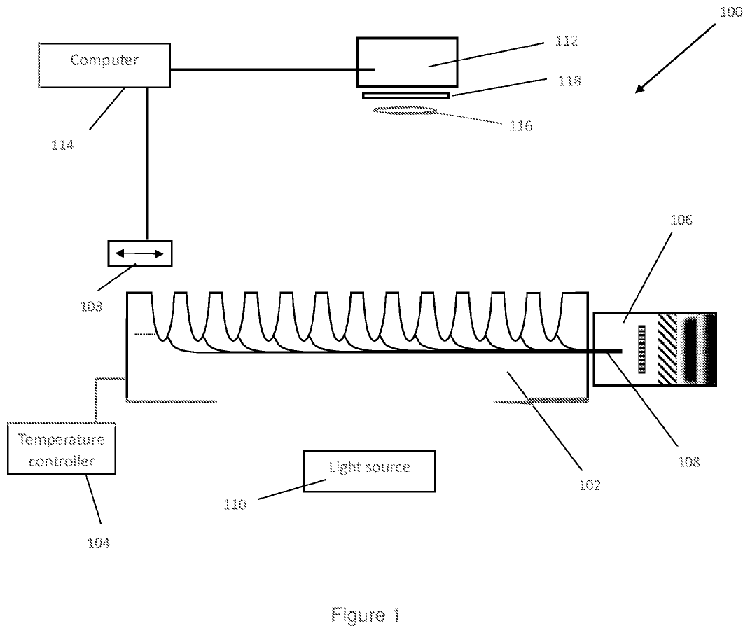

The system has a sample processing arrangement, in the described embodiments an incubator for holding and processing (incubating) a large number of samples such as a few hundred or thousand samples grouped in a number of groups of samples. The processing of the samples is controlled in a manner such that heating of each group of samples can be controlled individually. Further, the system comprises a detector and a light source and is arranged such that a change in an illumination property and/or a change in a detection property can transfer the system (or parts thereof) between fluorometric and colorimetric screening mode or between distinct fluorometric screening modes. A specific embodiment of the system will now be described with reference to FIG. 1.

FIG. 1 shows the screening system 100 to identify pathogens or genetic differences. The system 100 comprises an arrangement for processing samples, which in this embodiment is provided in the form of an incubator 102. The incubator 102 includes sample holders and is loaded and unloaded with samples by a robotic system (not shown).

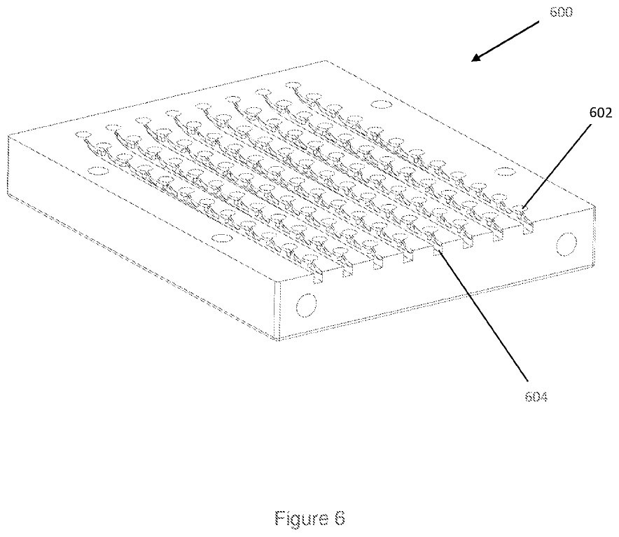

Each group of samples has in this example 96 individual sample holders for holding 96 individual samples. In this embodiment the incubator 102 includes sample holder blocks each arranged for holding one group of 96 individual samples. A sample holder block is shown in FIG. 6 and will be discussed further below in details. A person skilled in the art will appreciate that alternatively the sample processing arrangement may include any other number of sample holder blocks each having any suitable number of sample holders.

In the described embodiment the system 100 comprises sealed microplates with samples (not shown).

A person skilled in the art will appreciate that alternatively the system 100 may comprise other types of sample vessels instead of microplates, such as capillaries or tubes (which may be held in racks of a transparent material).

The system 100 further comprises a robotic system 103 for loading and unloading of samples into and out of the incubator 102. The robotic system 103 is controlled by a computer 114 and the system 100 is in this embodiment arranged to identify if and when the screening and/or processing is completed for individual samples or groups of samples (or microplates with samples). The robotic system 103 then removes the individual samples or groups of samples (or microplates with samples), which may be at random positions within the incubator 102 and may be surrounded by samples for which the screening and/or processing is not yet completed whereby vacant positions in the incubator are generated. Thereafter the robotic system 103 obtains fresh samples or groups of samples (or microplates with fresh samples), for example for a sample waiting station (not shown), and fills the vacant positions in the incubator 102 with the fresh samples. In this manner the system 100 allows continuous throughput of samples, which facilitates very high throughput not possible with a batch processing technique.

The system 100 comprises a source of electromagnetic radiation, which in this embodiment is provided in the form of light source 106. The light source 106 provides light for fluorometric screening and has LEDs that provide light having a wavelength required for exiting the emission of fluorescence emission by the samples. In a variation of the described embodiment the light source 106 may additionally or alternatively be arranged to provide illumination for alternative fluorometric or colorimetric measurements.

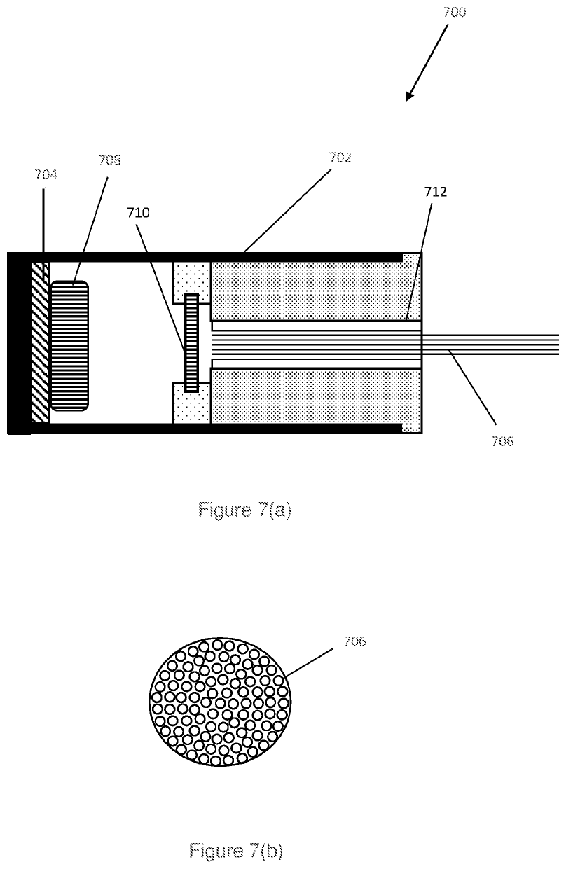

The light source 106 is coupled to the samples using an optical fibre bundle 108. Optical fibres of the optical fibre bundle 108 couple light from the light source 106 into individual sample holders and individual samples. The incubator 102 comprises in this example 32 sample holder blocks each having 96 sample holders each carrying a sample. The light source 106 is configurable and will be explained in detail further below with reference to FIGS. 7(a) and 7(b).

In the illustrated embodiment the system 100 comprises a further source of electromagnetic radiation, which is provided in the form of light source 110. The light source 110 is a broadband light source and provides light required for colorimetric screening and/or a secondary fluorometric screening. The light source 110 comprises filters and illuminates the samples from a position below the samples. In a variation of the described embodiment the light source 110 may also illuminate the samples from a position above the samples or from a horizontal direction.

The system 100 comprises a detector 112 which may be provided in different forms. In one embodiment the detector 112 is a colour camera, such as a suitable colour CCD camera. The colour camera is controlled by the computer 114 and is in this embodiment moveable over the sample holder blocks of the incubator 102. The movement of the detector 112 is also controlled by the computer 114 and screening may be conducted for a succession of selected sample holder blocks.

The detector 112 comprises a focusing lens 116 and a suitable filter 118. The detector 112 is arranged to receive light that transmitted through the samples from the light source 110 and can consequently be used for colorimetric measurements. The lens 116 focuses the samples onto an image plane of the detector 112 and it is possible to correlate locations of samples with an outcome of the colorimetric screening using suitable image processing software routines. Further, the detector 112 detects the fluorescence light emitted by the samples in response to the excitation light received from the light source 106. Again, it is possible to correlate locations of samples with an outcome of the fluorometric screening. In this manner it is possible to perform colorimetric and fluorometric measurements concurrently. Further, as the light source 106 is configurable, fluorometric screening may only be conducted for some samples or sample holder blocks.

In another embodiment the detector 112 is provided in the form of a monochrome detector. Again, the detector 112 has suitable filters. A first filter may allow transmission of light associated with colorimetric screening and a second filter may allow detection of fluorescence radiation. As the properties of the filters are known, it is possible to perform either colorimetric and/or fluorometric screening using the monochrome detector. The detector has a filter wheel that allows change of the filters in minimal time. The detector and the filter wheel are controlled by computer 114 and it is possible to conduct fluorometric and colorimetric measurement in close succession using the monochrome detector. The filters may be suitable long-pass or bandpass filters.

In a variation of the above-described embodiment the detector 112 may be a monochrome detector and comprises a multi-pass filter (instead of a filter wheel) having a first pass-band allowing the transmission of light at a wavelengths range required for colorimetric mode detection and a second pass-band allowing the detection of light at a wavelength range wavelengths range required for detection in the fluorometric mode. By transferring between illumination suitable for the colorimetric mode and illumination suitable for the fluorometric mode, the system may be transferred between the fluorometric mode (using light source 106 for example) and the colorimetric mode (using light source 110 for example) and the fluorometric and colorimetric measurements are possible in sequence using the detector with the multi-pass filter.

A further variation of the described embodiment relates to the detection in two different fluorometric screening modes. The detector 112 may be a monochrome detector or a colour detector and may comprise a suitable long-pass filter or band-pass filter. Dye molecules for the two different fluorometric screening modes may require excitation light at respective first and second wavelengths, but may have fluorescence emission that is within the pass-band of the band-pass filter of the detector or beyond a threshold wavelength of the long-pass filter of the detector. In this embodiment it is possible to transfer between both fluorometric detection modes by switching between a light source providing the excitation light at the first wavelength and a light source providing the excitation light at the second wavelength. Resulting images captured by the monochrome detector may be time-resolved to separate out the dye molecules excited by the first wavelengths and second wavelengths.

In a similar manner ratiometric measurements are possible. For example, ratiometric intensity measurement may require illumination of samples at a first wavelengths range and at a second wavelengths range. By selecting the illumination at the first wavelengths range and subsequently illumination at the second wavelength range (by choosing suitable filters for the light source 110 for example) and detecting respective light intensities using the monochrome detector, ratiometric intensity measurement are possible even if the detector is monochrome detector.

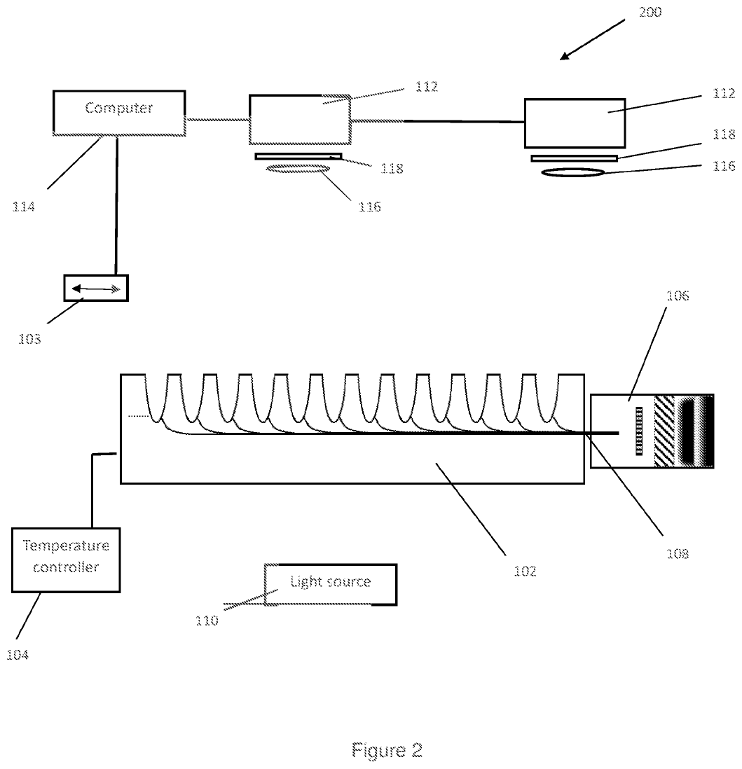

Turning now to FIG. 2, there is shown a screening system to identify pathogens or genetic differences in accordance with a further embodiment of the present disclosure. FIG. 2 shows the system 200 for screening of pathogens or genetic differences. The system 200 shown in FIG. 2 is related to the system 100 shown in FIG. 1 and like components are given like reference numerals. However, in contrast to the system 100, the system 200 has in this example 2 (or more) detectors 112. In one variation the detectors 112 are colour cameras. Each detector 112 maybe associated with a different area of the incubator and may concurrently screen different samples. If a relatively large number of detectors is used, the detectors 112 may not necessarily be moveable, but may be stationary each associated with a sample holder block of the incubator 102 (for example). Each detector 112 may be arranged for concurrent colorimetric and fluorometric screening or one or more detectors may be arranged for one screening mode while concurrently one or more other detectors 112 are arranged for the other screening mode.

Alternatively, at least one of the detectors 112 or each detector 112 may be monochrome detectors. In one specific embodiment the system 200 comprises a pair of monochrome detectors. One of the monochrome detectors has in this example a filter selected for colorimetric screening and the other has a filter selected for fluorometric screening whereby it is possible to perform fluorometric and colorimetric screening concurrently either for the same samples or for different samples (dependent on the position of the detectors). Optionally, one of the monochrome detectors has in this example a filter selected for a first fluorometric screening mode and the other has a filter selected for a second fluorometric screening mode whereby it is possible to perform the first and second fluorometric colorimetric screenings concurrently, either for the same samples or for different samples (dependent on the position of the detectors).

As the detectors are configurable, the detectors can be transformed between a colorimetric screening mode and a fluorometric screening mode. The pair of detectors maybe moveable to screen samples in different sample holder blocks in succession (for example). Alternatively, a relatively large number of detectors 112 is used and the detectors 112 may not necessarily be moveable, but may be stationary each associated with a sample holder block of the incubator 102 (for example).

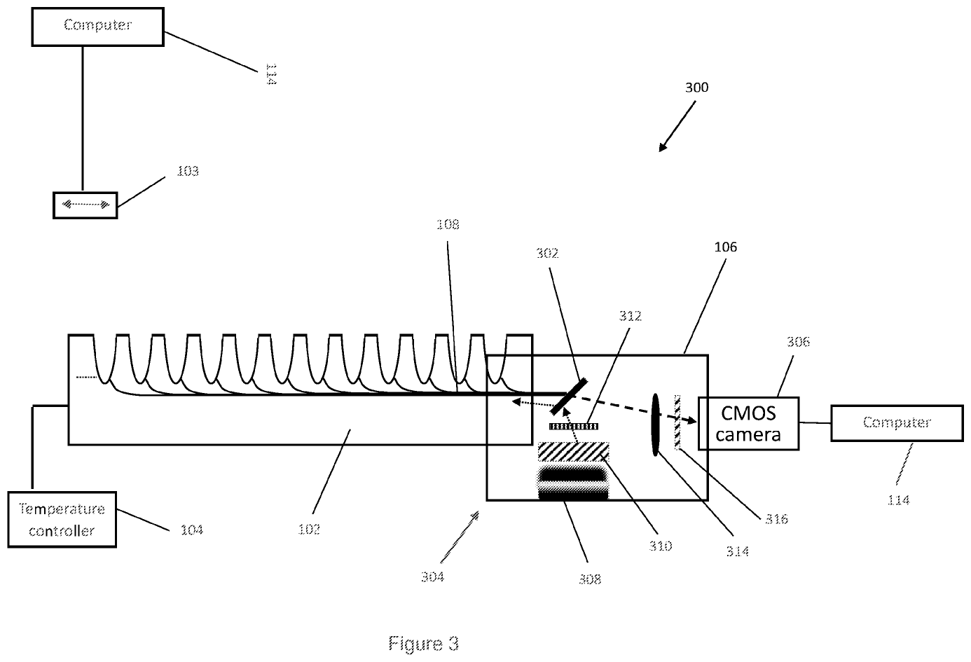

FIG. 3 shows a screening system to identify pathogens or genetic differences in accordance with another embodiment of the present disclosure. The system 300 is related to the system 200 shown in FIG. 2 and like components are given like numerals are given like reference numerals. The system 300 comprises a dichroic combiner/splitter 302 which optically couples light source 304 and detector 306 to the samples via optical fibres 108. The light source 304 comprises in this example a printed circuit board with LEDs 308, a concentrator lens 310 and an excitation filter 312. The detector 306, for example, a camera, is in this example a CMOS camera and receives light via a macro lens 314 and a long-pass filter 316. The optical fibres 108 serve a dual function. The optical fibres guide light (for example for fluorometric detection) from the light source 304 and the dichroic combiner/splitter to the samples in the incubator 102 and guide fluorescent light from the samples to the detector 306 again via the dichroic combiner/spitter 302. Optionally, the system 300 may comprise a further detector (not shown), such as the detector 112 shown in FIG. 1 (with lens 116, filter 118 and coupled to computer 114) and a further light source positioned below the incubator, such as the light source 110 which may be used for concurrent colorimetric screening.

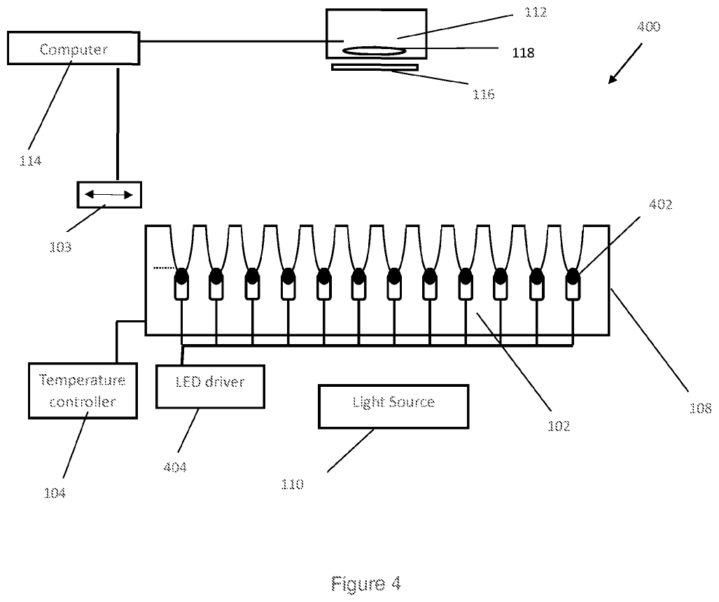

FIG. 4 shows a screening system to identify pathogens or genetic differences in accordance with a further embodiment of the present disclosure. The system 400 is related to the system 100 and like components are given like reference numerals. The system 400 comprises in this embodiment LEDs 402 which are positioned at sample holders for receiving samples. In the illustrated embodiment one LED is positioned at a respective individual sample holder, but in a variation of the illustrated embodiment each LED may also be associate with a group of sample holders or more than one LED may be positioned at each sample holder. The LEDs are controlled by LED driver 404 and are each equipped with suitable filters arranged to further narrow the emission wavelength band of the light emitted by the LEDs. In this embodiment the LEDs 402 are used to generate light for exciting fluorescence emission for fluorometric screening and the fluorescence emission is detected by the detector 112. Concurrent colorimetric screening or secondary fluorometric screening is possible using light source 110. In a variation of the described embodiment the system 400 may also comprise more than one detector (monochrome or colour) as described above with reference to FIG. 2.

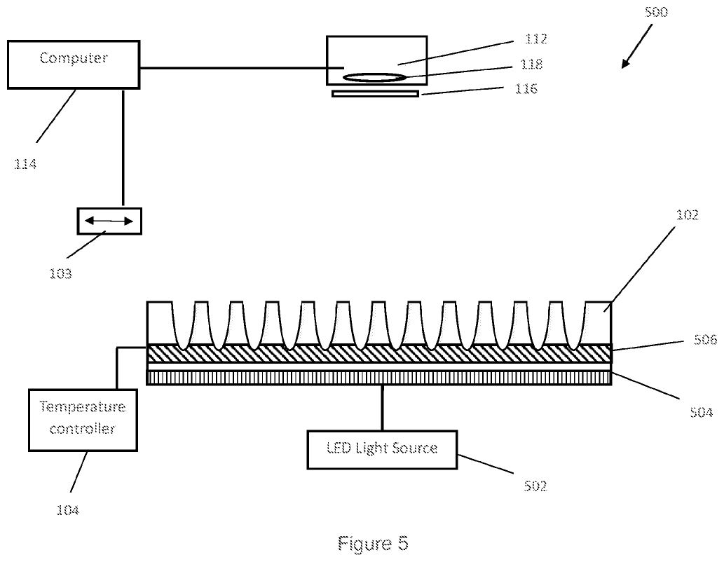

FIG. 5 shows a screening system to identify pathogens or genetic differences in accordance with another embodiment of the present disclosure. The system 500 is related to the system 400 and like components are given like reference numerals. The system 500 comprises a light diffuser 504 and a filter 506 arranged to further narrow the emission wavelength band of the light emitted by the LEDs. Optically coupled to diffuser 504 is the LED light source 502. The LED light source 502 comprises a plurality of LEDs that are coupled to one or more minor sides (edges) of the diffuser 504 or to an underside of the diffuser 504 so that the LEDs can emit light into the diffuser 504. The LEDs are controlled by LED driver (not shown). In this embodiment the LEDs of the light source 502 are used to generate light for exciting fluorescence emission for fluorometric screening and the fluorescence emission is detected by the detector 112. In a variation of the described embodiment the system 400 may also comprise more than one detector (monochrome or colour) as described above with reference to FIG. 2.

The embodiments depicted in FIGS. 1-5 show that there are numerous ways and configurations the disclosed screening system 100, 200, 300, 400 and 500 may be operated. These different ways and configurations allow the use of various screening modes. The following are some examples of screening modes that may be used, either in isolation or combination.

### Mode 1

Fluorescence resonance energy transfer (FRET) as typically used in multiplexed LAMP screening. For example, one FRET dye system can be excited in the shorter UV to blue wavelengths, and an emission in the green wavelengths can be detected. A second FRET dye system can be excited in the green wavelength and corresponding emission fluorescence measured in the yellow region of the spectrum. Similarly, excitation of a third FRET fluorophore in the yellow-orange region of the spectrum could stimulate emission which can be detected in the red to far-red wavelengths. In an embodiment, a single FRET donor is used, such as Syto-9, and a plurality of FRET acceptors with overlapping emission spectra but distinct emission spectra can be used. For example, a single FRET donor can be used to with a first, second and third FRET acceptor that each are excited in the green wavelengths, where the first FRET acceptor emits in the yellow wavelengths, the second FRET acceptor emits in the orange wavelengths, and the third FRET acceptor emits in the red wavelengths.

Multiplex Dye systems compatible with this mode for LAMP FRET include those using Molecular Beacon, DARQ and the MD-LAMP system.

### Mode 2

This Mode uses a first non-specific fluorophore dye that fluoresces strongly only once bound non-specifically to double stranded nucleic acid. In an embodiment, the binding is through being a minor groove binder and which is excited in the UV to violet and/or the indigo to blue wavelengths, and emits light in a longer wavelength, such as within the green or orange spectrum. Examples of this first sequence non-specific ‘donor’ dye include the green fluorescing minor groove biding dye Syto-9, or alternatively the orange-fluorescing non-specific dye Syto-82. This first non-specific dye acts as an energy donor in each case.

A second, dye (the FRET acceptor) fluorophore is excited via FRET energy transfer from an overlapping (i.e., green, in the case of Syto-9, or orange in the case of Syto-82) emission spectrum from the first non-specific dye. This second sequence-specific FRET acceptor probe incorporates a fluorophore chosen to have a longer emission wavelength emission from the first donor fluorophore (emitting for example in yellow area of the spectrum when paired with Syto-9 as the FRET donor) is incorporated into a sequence-specific oligonucleotide primer of a nucleic acid amplification reaction (preferably at the 5′ end), such that it only fluoresces as the amplification product accumulates, bringing the more of the minor groove binding dye in close proximity to the acceptor dye to allow detection using the detector. The second dye in this case may include, for example, Dy-Light 509/590, 6-ROX (6-Carboxy-X-rhodamine), Dy-515-LS, Dy-521-LS, and Alexafluor 594, Dy-594, Texas Red, Star Orange, iFluor594, eFluor-610.

Alternatively or in addition, another minor groove binding dye Syto-64, Syto-82 or Sytox-Orange may be excited by direct illumination or by proximal fluorescence of a dye in the blue or green region of the spectrum and may transfer its energy via FRET to an acceptor fluorophore (the excitation wavelength of which overlaps with the yellow/orange emission frequency of the minor groove DNA binding dye), incorporated into a sequence-specific oligonucleotide primer of a nucleic acid amplification reaction (preferably at the 5′ end), such that the acceptor fluorophore is only excited sufficiently by FRET to allow fluorescence as the amplification product accumulates, bringing the more of the minor groove binding dye in close proximity to the acceptor dye, allowing activation via FRET and resulting fluorescence in the red region of the spectrum. Examples of suitable red-emitting fluorophore acceptor dyes with FRET excitation spectra which overlap with the yellow/orange spectral range include: NovaFluor 685, Cy5c, Cy5.5c, LC Red 640e, CAL Fluor Red 635, LC Red 670e, Quasar 670, Oyster 645d, LC Red 705e, Y578, Alexofluor-647 Alexafluor 660, and Atto-655, Sytox-DeepRed, Atto 665, HiLight647.

Those skilled in the art would understand that the FRET dyes chosen for this this Mode would need to be matched to ensure there is appropriate overlap (preferably >30%) between the FRET donor emission wavelength and the FRET acceptor excitation wavelengths. The proximity condition (i.e., the donor and acceptor molecules being within 10 nM of one another) is met by random non-specific incorporation of some of the donor dye molecules close to the acceptor. The principles governing the choice of each pair of dyes from the above list are reviewed here: Bajar, B. T., Wang, E. S., Zhang, S., Lin, M. Z. & Chu, J. A Guide to Fluorescent Protein FRET Pairs. Sensors 16, 1488 (2016).

An advantage of this Mode is that it can be operated using common dyes/probes without the need to design additional specific FRET dye systems of Mode 1. For example, the FRET acceptor dye does not have to be a self-quenching dye, nor does it have to displace the LAMP amplicon product. It can instead simply be a labelled form of one or more of the standard LAMP primers.

For example, a green non-specific FRET donor dyes Syto-9 could be paired with a sequence specific probe with a narrow yellow fluorescence spectrum. Likewise, the orange non-specific dyes Syto-64 or Syto-82 could be paired with a dye fluorescing in the far-red spectrum, such as NovaFluor 685.

### Mode 3

Use a dye with a fluorophore that is excited in the NIR/IR/far-red but has an emission that is upconverted to the yellow-red wavelengths. The detector used to detect the emission would use a NIR/IR/far-red filter to block out the wavelength(s) used for excitation. The NIR/IR/far-red filter may be integrated into a colour or monochromatic camera.

### Mode 4

Use two (or more) dyes/probes that have different excitation wavelengths but similar emission wavelengths. This Mode uses one or more sources of electromagnetic radiation to provide the different excitation wavelengths. For example, a single multiwavelength source of electromagnetic radiation may be used in conjunction with a multi-pass filter to provide first and second excitation wavelengths. Optionally, two different electromagnetic radiation sources with fixed excitation wavelengths may be used. As the emission wavelengths are similar, a single detector may be used for detection, which can help to reduce complexity of the system. To correlate the emission wavelengths with the excitation source, and thus what dye/probe data is being captured, the data captured by the detector is time-resolved to correlate the emission data with the associated dye/probe. For example, a first excitation wavelength is provided to excite a first dye and the emission from this first dye is captured, and then a second excitation wavelength is provided to excite a second dye the emission from this second dye is captured. The excitation wavelengths are switched between the first and second excitation wavelengths.

The dyes/probes used in this Mode may be that of another Mode, such as Mode 1 or Mode 2 to utilise different excitation wavelengths.

### Mode 5

Use two (or more) dyes/probes that excite at the same or similar wavelengths, but that emit at different wavelengths. The detector used in this Mode would be configured to detect different emission wavelengths. The detector may by a monochromatic detector or may be a multi-wavelength detector.

### Mode 6

Use standard LAMP probes, such as single excitation/emission dyes that only emit once hybridised occurs, e.g., dyes that self-quench.

### Combination of Modes

The above Modes can act as the first and/or second screening mode. The above Modes may also be used in combination. For example, Mode 1 (or Mode 2) and Mode 3 could be performed concurrently. For example, a UV light could be used as the source of electromagnetic radiation for Mode 1 or Mode 2, and a NIR/IR/far-red light could be used as the source of electromagnetic radiation for Mode 3. If the wavelengths of the emissions of the different dyes/probes are different, a single detector such as a colour camera may be used to detect the emission wavelengths, or separate detectors configured to detect each emission wavelength could be used. However, if the wavelengths of the emissions are similar or the same (as per Mode 4), a single detector can be used such as a monochromatic camera, but the excitation sources would be switched on and off and the data collected by the detector would be time-resolved with the respective excitation source. Using a single excitation source or detector can help to reduce the complexity of the system and may help to increase throughput rate. However, even using multiple excitation sources that require switching on and off, rather than changing of optical properties such as adjusting optical filters, can provide an increase in throughput rate compared to existing systems.

An advantage of Mode 5 is that only a single source of electromagnetic radiation is required. If the emission wavelengths used in Mode 5 are constant, for example one emission from one dye in the green and another emission from another dye in the red, a single detector such as a colour camera could be used or two separate monochromatic cameras for detecting either green or red. Using monochromatic cameras can help to eliminate the need for filters, which can help to reduce complexity and increase throughput rate.

A plurality of monochromatic cameras can be grouped together or “stacked” to form a single detector. An advantage of a stacked detector is that there is no need to activate or switch filters, and instead any switching can be performed electronically by the computer, 114 which can lead to higher throughput rates and reduced complexity.

The source of electromagnetic radiation used in Modes 1-6 may be a fixed wavelength source, or a multi-wavelength source. A combination of fixed and multi-wavelength sources may be used. Multiple wavelength source may include tuneable lasers, different LEDs, use of optical filters and mirrors, and/or use of dichroic filters.

Similarly, the detectors may be a fixed wavelength detector, or a multiwavelength detector. Fixed wavelength detectors include monochromatic cameras. Multiwavelength detectors may include the use of multi-pass filters, multiple cameras working concurrently such as separate red, green, and blue cameras, photodiode arrays, single pass filters, and/or colour cameras. In an embodiment, each well of the plurality of samples has its own detector. For example, in a 96-well plate, 96 detectors, such as separate photodiode arrays, are used for detecting emission in each well. Such an embodiment may be used in solid-state continuous monitoring. Multiple photodiode arrays may be associated with each well. An advantage of a photodiode array such as a photomultiplier is that they can have in-built filters which can eliminate the need for addition of filters, helping to reduce complexity and increase throughput rate because there is no need to synchronise filtering with data capture.

In embodiments where a single source of electromagnetic radiation is used or a single detector is used, the computer 114 controls the source of electromagnetic radiation or detector to time-resolve the source of electromagnetic radiation and the resulting emission or colorimetric data from the detector.

In an embodiment, the detector 112, for example, a camera, captures an image of a whole plate rather than imaging individual wells. A visual reference datum in the incubator 102 can be used to orientate the captured image of the plate relative an orientation of the plate thereby ensuring locations of individual wells can be identified.

An issue with existing high-throughput systems is that they rely on the use of switchable/moveable components such as filters and the like that need to be synchronised with a source of electromagnetic radiation, e.g., excitation source and/or detector used to detect, e.g., emission wavelength(s). This switching and synchronisation means a maximum throughput rate is limited to about 1000 samples per hour. In addition, the requirement for switchable filters and synchronisation means that the system is complex and expensive. For example, any time mechanical movement is required, the time required for mechanical movement is multiplies hundreds to thousands of times, which can have a significant impact on the throughput rate.

In contrast, an embodiment of the current disclosure does not rely on switchable filters. For example, embodiments utilising Modes 1-6 above can be operated with fixed filters, such as using a monochromatic camera for detection, meaning an optical system associated with the detector and/or electromagnetic source does not need adjusting “on the fly” during use of the system. The inventors have found that using fixed filters instead of switchable filters, and elimination or reducing synchronisation, can increase a throughput rate to be at least 2000 samples per hour, such as >4000 samples/hour. The minimal use of filters allows for a system that is less complex, leading to a more robust system that is easier and cheaper to operate.

Turning now to FIG. 6, an embodiment of a sample holder block of the incubator 102 is now described in further detail. The incubator 102 comprises a plurality of the sample holder blocks 600 and each sample holder block 600 is connected to the temperature controller 104 shown in FIGS. 1 and 2 such that the heating of each sample holder block 600 can be individually controlled. For example, the sample holder block may include thermal cycling heater or may be arranged to heat at a fixed temperature. The sample holder block 600 comprises 96 individual sample holders 602 for receiving samples. Below each individual sample holder is a through hole to a groove 604. Each through hole is arranged to receive an optical fibre and the grooves 604 are arranged to receive bundles of the optical fibres, which are directed to the light source 106 shown in FIGS. 1, 2 and 3. The optical fibres emit in use light for excitation of fluorescence transitions for fluorometric screening. Alternatively, the optical fibres may in use also light for colorimetric screening into the individual sample holders.

Turning now to FIG. 7(a), there is shown a light source 700 according to an embodiment of the present disclosure. The light source 700 corresponds to the light source 106 shown in FIGS. 1 and 2. The light source 700 comprises a housing 702 and LEDs 704. The LEDs are arranged to emit light at wavelength as required by the fluorometric and colorimetric screening. The light emitted by the LEDs is selectable by selection which LED is operated. The LED light is coupled into optical fibres using collimator 708 and filter 710. A bundle of the optical fibres comprises in this example 96 individual optical fibres and is held in position by Ferrule 712. Insert FIG. 7(b) is a cross-sectional representation of the optical fibre bundle.

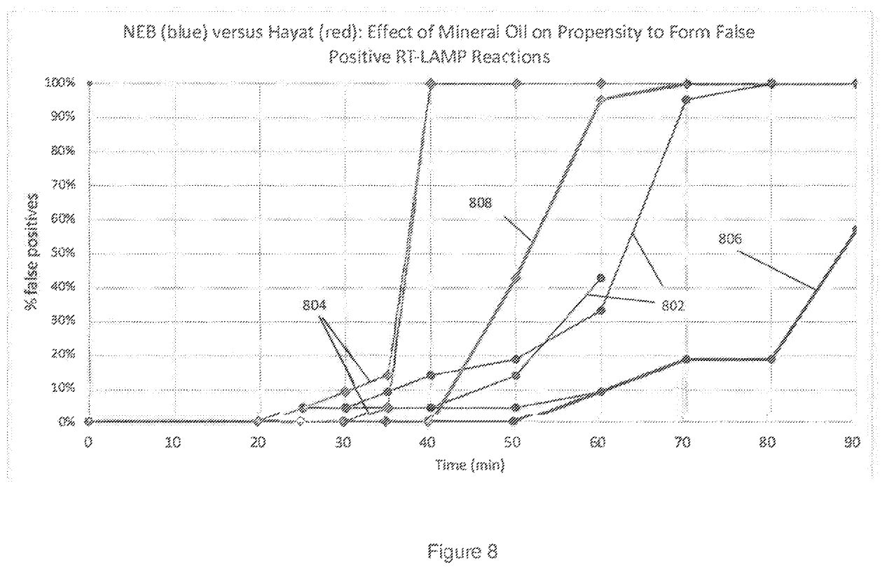

Embodiments of the system 100, 200 and 300 described above include sample vessels provided in the form of microplates with cavities, such as 96 cavities, for receiving 96 samples and which contain chemicals required for screening and/or processing of the samples. The cavities of the microplates are sealed. In one specific embodiment the cavities include an amount of a mineral oil. The inventors have observed that the presence of the mineral oil in the cavities has significant practical advantages for screening and processing of the samples as will be described below with reference to FIG. 8. FIG. 8 is a graph of false positives as a function of incubation time. The graph illustrates the effect of a layer of mineral oil on samples in each cavity (well) of a microplate with 96 samples using two different RT-LAMP chemistries-New England Biolabs (802 with oil layer and 804 without oil layer) and Hayat Genetics chemicals (806 with oil layer and 808 without oil layer). The graphs for the samples with oil layer (802, 806) show that because of the oil layers false positives can be either entirely avoided (using Hayat Genetics chemicals) or at least largely avoided (New England Biolabs chemicals) during a typical 30-minute RT-LAMP reaction time.

The inventors conclude that the oil layer reduces evaporation during the reaction which increases the concentration of components like primers and salt, both known to be associated with non-specific reactions between the RT-LAMP primers, if at concentrations which are too high. Using the oil layer it is consequently possible to extend the incubation period for the RT-LAMP reaction longer (allowing more time for real positives to emerge), before moving into a ‘danger zone’ where false positives arise.

In summary, the use of mineral oil layers in RT-LAMP reactions has the following (further) advantages:

An unexpected improvement in consistency and quality of fluorescent signals. The inventors speculate that this may be due to a ‘lensing’ effect of the oil droplet;
avoidance or reduction of frequency of false positives arising early in the incubation period (i.e eliminated within first 30 minutes of 65 degree incubation for Hayat Genetics chemistry) for RT-LAMP reactions; and
for colorimetric RT-LAMP reactions, the oil provides a seal which blocks unwanted aeration of the reaction mix as excessive exposure to air can cause colorimetric RT-LAMP reactions to spontaneously acidify (from dissolved CO₂ making carbonic acid). This phenomenon confounds the reaction readout (which monitors acidification of pH).

Further, the use of the oil layer in each cavity of a microplates (for example) makes the microplate (with the chemicals for processing the samples in the cavities) more stable for shipment and storage (eg. at −20° C.). In addition, the oil layer improves the quality of results from colorimetric and fluorometric RT-LAMP reactions, reducing the false positive rate.

A person skilled in the art will appreciate that variations of the described embodiments are possible. For examples, the incubator may comprise any number of sample holder blocks. Further, each sample holder block may comprise any number of sample holders. In another variation the incubator may not necessarily comprise sample holder blocks and individual sample holders may be arranged in any other suitable manner. In addition, the system may be suitable for processing any number of samples and may comprise any number of detectors and sources of electromagnetic radiation. The system may alternatively also be arranged for screening using other modes, such as luminescence or phosphorescence screening modes.

Reference that is being made to prior art publication is not an admission that the prior art publications are part of the common general knowledge of a skilled person in Australia or another country.

The preferred embodiments of the disclosure have been described above to explain the principles of the present disclosure and its practical application to thereby enable others skilled in the art to utilize the present disclosure. However, as various modifications could be made in the constructions and methods herein described and illustrated without departing from the scope of the present disclosure, it is intended that all matter contained in the foregoing description or shown in the accompanying drawings, including all materials expressly incorporated by reference herein, shall be interpreted as illustrative rather than limiting. Thus, the breadth and scope of the present disclosure should not be limited by the above-described exemplary embodiment but should be defined only in accordance with the following claims appended hereto and their equivalents.

1. A screening system to identify pathogens or genetic differences, wherein the system has a first and second screening mode and comprises:
a source of electromagnetic radiation for illuminating a plurality of samples, the source of electromagnetic radiation having a selectable illumination property; and
a detector for detecting electromagnetic radiation transmitted through or emitted by the plurality of samples, the detector having a selectable detection property; and
an incubator for incubating the samples;
a robotic system for loading and unloading of samples;
wherein the system for screening of pathogens or genetic differences is arranged to identify if and when the screening and/or processing is completed for individual samples or groups of samples, and wherein the robotic system is arranged to:
remove the individual samples or groups of samples from the incubator leaving vacant sample holders or groups of sample holders, wherein samples or groups of samples are being removed from locations surrounded by, or adjacent to, samples or groups of samples for which screening and/or processing is not completed; thereafter
obtain fresh samples or groups of samples; and thereafter
fill the vacant positions in the incubator with the fresh samples;
whereby the system is suitable for continuous throughput of samples.
wherein the system is arranged for operation in the first and second screening mode during incubating of the samples in the incubator.

2. The system of claim 1 wherein the system is arranged for concurrent or quasi-concurrent operation in the first and in the second mode.

3. (canceled)

4. (canceled)

5. The system of claim 1 wherein the first screening mode is a fluorometric screening mode and the second screening mode is a second fluorometric screening mode or colorimetric screening mode.

6. The system of claim 1 wherein the system is arranged such that individual samples or individual groups of samples can be concurrently screened using different conditions.

7. The system of claim 1 wherein the arrangement for the incubator comprises heaters and one or more controller enabling individual control of heating of individual samples or individual groups of samples.

8. (canceled)

9. (canceled)

10. (canceled)

11. The system of claim 1 wherein the source of electromagnetic radiation comprises a light source for the fluorometric mode and a light source for the colorimetric mode.

12. The system of claim 1 wherein the source of electromagnetic radiation comprises individual light elements with filters which are positioned at respective sample holders for direct illumination of the samples.

13. (canceled)

14. The system of claim 1 comprising optical fibres between the source of electromagnetic radiation and individual sample holders or groups of the sample holders and wherein the optical fibres are guided through portions of the arrangement for processing the samples to the individual sample holders or to groups of the sample holders.

15. (canceled)

16. The system of claim 1 wherein the detector is one of a plurality of detectors and wherein at least two detectors are monochrome detectors.

17. (canceled)

18. The system of claim 1 comprising optical fibres between the detector and each individual sample holder or group of the sample holders, wherein the optical fibres are positioned to receive radiation from the samples and direct the received radiation to a suitable a detector.

19. (canceled)

20. The system of claim 1 wherein the detector is moveable to detect electromagnetic radiation at a location near an individual sample or group of individual samples and wherein movement of the detector is controlled by a controller.

21. A screening system to identify pathogens or genetic differences, wherein the system has a first and second screening mode and comprises:
a source of electromagnetic radiation for illuminating a plurality of samples, the source of electromagnetic radiation having a selectable illumination property; and
a detector for detecting electromagnetic radiation transmitted through or emitted by the plurality of samples, the detector having a selectable detection property; and
an incubator for incubating the samples;
a robotic system for loading and unloading of samples, wherein the system for screening pathogens or genetic differences is arranged to identify if and when the screening and/or processing is completed for individual samples or groups of samples, and wherein the robotic system is arranged to:
remove the individual samples or groups of samples from the incubator leaving vacant sample holders or groups of sample holders, wherein samples or groups of samples are being removed from locations surrounded by, or adjacent to, samples or groups of samples for which screening and/or processing is not completed; thereafter
obtain fresh samples or groups of samples; and thereafter
fill the vacant positions in the incubator with the fresh samples;
whereby the system is suitable for continuous throughput of samples.
wherein the system is arranged for transferring between the first screening mode and the second screening mode by selecting at least one of the detection property of the detector and the illumination property of the source of electromagnetic radiation during incubation of the samples in the incubator.

22. The system of claim 21 wherein the system enables operation in one of the first and second mode immediately after operation in the other one of the first and second mode.

23. (canceled)

24. The system of claim 21 wherein the system is arranged such that individual samples or individual groups of samples are screened using different conditions.

25. The system of claim 21 wherein, the arrangement for processing the samples comprises heaters and one or more controller enabling individual control of heating of individual samples or individual groups of samples.

26. (canceled)

27. (canceled)

28. (canceled)

29. (canceled)

30. (canceled)

31. The system of claim 21 wherein the detector is a monochrome detector comprising first and second filters and wherein the first filter allows transmission of electromagnetic radiation at a wavelengths range required for the fluorometric mode and the second filter allows transmission of electromagnetic radiation at a wavelengths range required for the colorimetric mode wherein the system is arranged for transfer between the fluorometric mode and the colorimetric mode by transferring between the first and second filter.

32. The system of claim 21 wherein the detector is a monochrome detector comprising a multi-pass filter having pass-windows allowing transmission of electromagnetic radiation at a wavelengths range required for the fluorometric screening mode and transmission of electromagnetic radiation at a wavelengths range required for the colorimetric mode, and wherein the system is arranged for transfer between the fluorometric mode and the colorimetric mode by transferring between illumination suitable for the colorimetric mode and illumination suitable for the fluorometric mode.

33. (canceled)

34. The system of claim 21 wherein the source of electromagnetic radiation comprises individual light elements with filters which are positioned at respective sample holders for direct illumination of the samples.

35. (canceled)

36. The system of claim 21 comprising optical fibres between the source of electromagnetic radiation and individual sample holders or groups of the sample holders and wherein the optical fibres are guided through portions of the arrangement for processing the samples to the individual sample holders or to groups of the sample holders.

37. The system of claim 21 comprising optical fibres between the detector and each individual sample holder or group of the sample holders, wherein the optical fibres are positioned to receive radiation from the samples and direct the received radiation to a suitable a detector.

38. (canceled)
